(19) United States
(12) Patent Application Publication
(10) Pub. No.: US 2011/0202009 A1
(43) Pub. Date: Aug. 18, 2011

(54) I.V. tubing safety strap

(76) Inventor: Sunil Persaud, Lewisville, TX (US)

(21) Appl. No.: 12/658,752

(22) Filed: Feb. 16, 2010

(51) Int. Cl. A61M 39/00

(52) U.S. Cl. 604/179

(57) ABSTRACT

A strap that is both easy to use and safe for both the patient and healthcare provider. A slip-on circular elastic strap is provided in various sizes in order to accommodate the different arm circumferences of patients. The circular strap has an additional small elastic strap affixed via stitching. The small elastic strap utilizes a hook and loop system such that the ends of the small strap can criss-cross over the I.V. tubing and hold the tubing in place to the circular elastic strap thus securing the I.V. tubing with tension preventing dislocation of the tubing and/or I.V. site.

### FIELD OF THE INVENTION

The present invention relates to I.V. tubing straps, more particularly to a strap that's easy to use and stretchable; allowing the patient/and or health care provider to reposition the strap for comfort. This method also allows the IV tubing to be secured preventing injury to the patient.

### BACKGROUND OF THE INVENTION

The traditional method for securing an IV has been the use of either plastic or paper adhesive tapes. The problems related to the use of adhesive tapes are skin tears which can lead to infections, and pain upon removal of tape; especially with hairy patients. Another complication is related to the overall security of adhesives. Over a period of time, the adhesive on the tape loses its properties, leading to eventual replacement of the adhesive tape and possibly compromising the stabilization of the I.V. site due to a lack of security. Skin variations such as oily skin, excessive body hair, and medical conditions such as psoriasis or eczema can further undermine the effectiveness of adhesives compromising the integrity of the I.V. site.

The IV tubing sure strap, patened in 1997, was invented with special attention to secure the Y-portion of the IV tubing making it easier to access the Y-port for administration of medications or other solutions. The IV safety cuff invented in 2008 was meant to address the damage caused by adhesive tape but still requires the use of adhesive tape to secure IV tubing to the product.

Currently IV tubing is secured with layers of adhesive tape which causes more harm to the patient including skin tears, allergic reactions, and pain from pulling of hair on the patient's arm. According to patient safety authority.org, a case was cited where adhesive tape led to a 8 cm×8 cm skin tear on a patients forearm from removal of adhesive tape from an infiltrated I.V. site, which later required suturing to repair the skin tear: PA PSRS patient Saf Advis 2006 September: 3 (3); 1, 5-10. Due to lack of breathability, adhesive tape can also harbour dirt and debris leading to potential bacterial infection. The 1997 patented IV tubing strap does not ensure the prevention of movement of the IV tubing, it does not stop bacterial infections, nor does it ensure comfort, adjustibility, or reusibility. It does not address the safety issue of the patient and is geared more towards the administration of medications by the healthcare professional. It also fails to adjust for the patients needs or body variations, compromising safety and comfort. The IV safety cuff invented in 2008 addresses the damage caused by adhesive tape, but still uses adhesive tape on their product as a means of securing IV tubing to the product; and securing the product once adjusted to provide a snug fit. It also claims to provide breathability of the skin underneath but has a potential to cause maceration or moisture build up leading to compromised skin integrity. It claims to be adjustable, but due to its bulky design, it covers a large surface area of the skin.

### SUMMARY OF THE INVENTION

In accordance with the present invention, provided is a strap that is both easy to use and safe for both the patient and healthcare provider. A slip-on circular elastic strap is provided in various sizes in order to accommodate the difference in arm circumferences. The circular strap has an additional small elastic strap; the small elastic strap utilizes a hook and loop system such that the ends of the small strap can criss-cross over the I.V. tubing and secure the tubing in place to the circular elastic strap which secures the I.V. tubing with tension; but not so much tension that it restricts the flow of the I.V. solution.

It would be advantageous to provide an elastic strap that secures I.V. tubing to a patient.

It would be advantageous to provide an I.V. tubing securing strap that does not utilize adhesive.

It would be advantageous to provide an I.V. strap that is reusable and easy to use for both the healthcare provider and patient.

It would further be advantageous to provide an I.V. tubing securing strap that is comfortable and can stabilize I.V. tubing while preventing skin irritation or damage.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a front perspective with application detail view of an elastic i.v. tubing safety strap in accordance with the invention; and

FIG. 2 is an exploded and frontal view view of an elastic i.v. tubing safety strap in accordance with the invention.

For purposes of clarity and brevity, like elements and components will bear the same designations and numbering throughout the Figures.

### DESCRIPTION OF THE PREFERRED EMBODIMENT

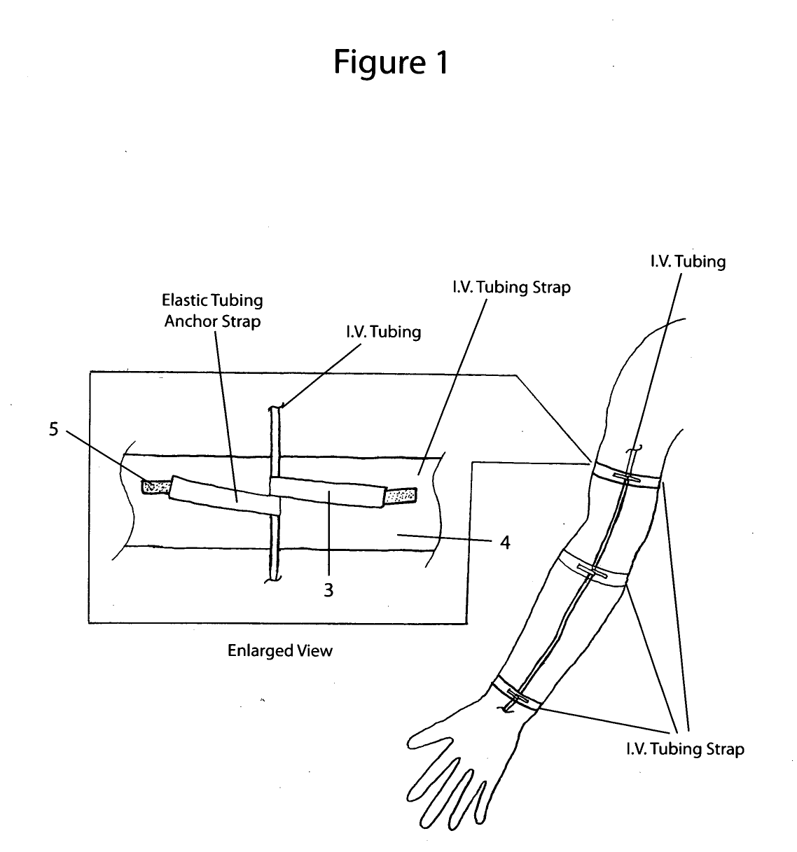

FIG. 1 is a front perspective with application detail view of an elastic i.v. tubing safety strap in accordance with the invention.

The circular i.v. tubing strap 4 is shown applied to patient's arm with the I.V. tubing in its appropriate application. The rectangular elastic tubing anchor strap 3 shown has a loop system 6 in the form of a square affixed to each end of the rectangular elastic tubing anchor utilizing a strong adhesive. The rectangular elastic tubing anchor strap 3 is stitched at its midpoint to the circular I.V tubing safety strap, enabling the elastic tubing anchor strap 3 to overlap the I.V. tubing in a criss-cross fashion and provide tension when stretched and affixed to the i.v. tubing strap 4 utilizing the hook system 5 which is attached to it, centered behind the ends of the elastic tubing anchor strap 3.

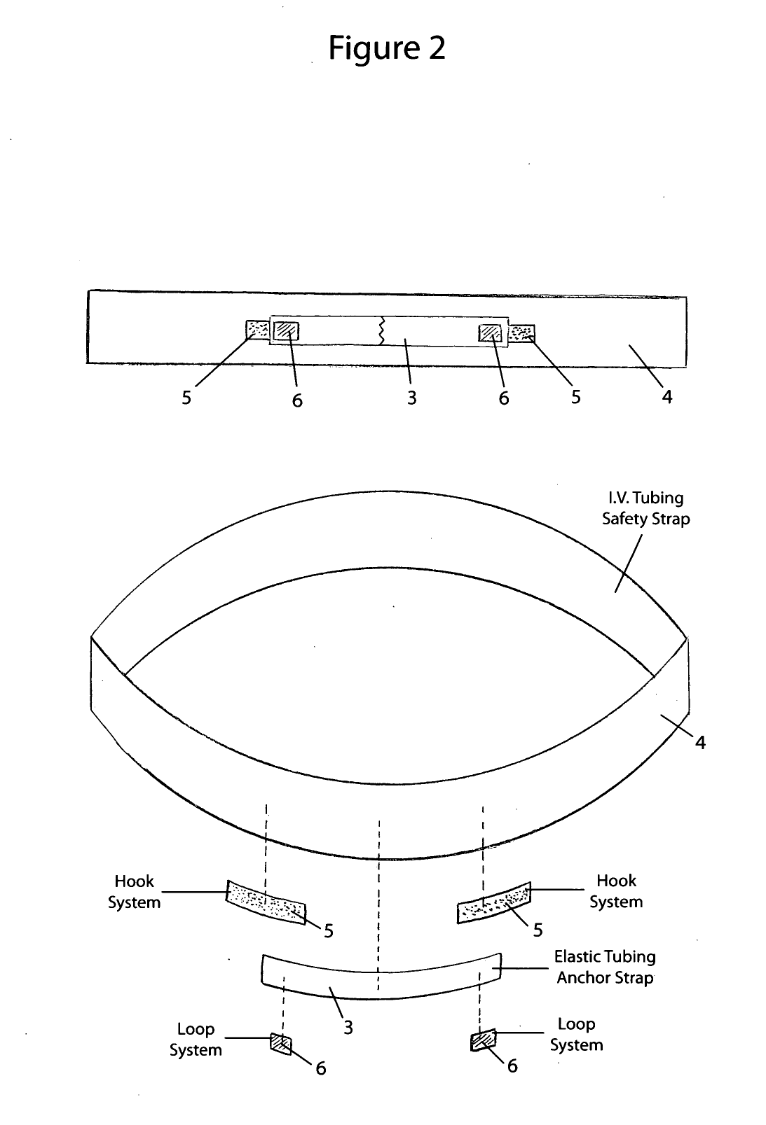

FIG. 2 is an exploded and frontal view of an elastic i.v. tubing safety strap in accordance with the invention. The elastic tubing anchor strap 3 is stiched onto the i.v. tubing strap 4. The hook system 5 is adhered to the i.v. tubing strap 4 and the loop system 6 is adhered onto the ends of the elastic tubing anchor strap 3.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. An i.v. tubing safety strap for securing i.v. tubing onto a patient without the use of adhesives; preventing pain and skin damage or exacerbation of pre-existing skin conditions, comprising:
means for securing the i.v. tubing onto the strap providing stability and minimizing movement;
means for providing a strap that is easy to apply onto a patient for the purpose of mounting i.v. tubing, threadably attached to said means for securing the i.v. tubing onto the strap providing stability and minimizing movement;
means for providing a surface to which the ends of the elastic tubing anchor strap can attach, securely adhered to said means for providing a strap that is easy to apply onto a patient for the purpose of mounting i.v. tubing, and adhesively connected to said means for securing the i.v. tubing onto the strap providing stability and minimizing movement; and
means for providing a surface that will attach to the hook system allowing the elastic tubing anchor strap to secure the i.v. tubing, securely adhered to said means for securing the i.v. tubing onto the strap providing stability and minimizing movement.

2. The i.v. tubing safety strap in accordance with claim 1, wherein said means for securing the i.v. tubing onto the strap providing stability and minimizing movement comprises an elastic/stretchable, rectangular elastic tubing anchor strap.

3. The i.v. tubing safety strap in accordance with claim 1, wherein said means for providing a strap that is easy to apply onto a patient for the purpose of mounting i.v. tubing comprises a circular, elastic/stretchable i.v. tubing strap.

4. The i.v. tubing safety strap in accordance with claim 1, wherein said means for providing a surface to which the ends of the elastic tubing anchor strap can attach comprises a rough texture, rectangular, flexible hook system.

5. The i.v. tubing safety strap in accordance with claim 1, wherein said means for providing a surface that will attach to the hook system allowing the elastic tubing anchor strap to secure the i.v. tubing comprises a soft surface, looped low pile material, square loop system.

6. An i.v. tubing safety strap for securing i.v. tubing onto a patient without the use of adhesives; preventing pain and skin damage or exacerbation of pre-existing skin conditions, comprising:
an elastic/stretchable, rectangular elastic tubing anchor strap, for securing the i.v. tubing onto the strap providing stability and minimizing movement;
a circular, elastic/stretchable i.v. tubing strap, for providing a strap that is easy to apply onto a patient for the purpose of mounting i.v. tubing, threadably attached to said elastic tubing anchor strap;
a rough texture, rectangular, flexible hook system, for providing a surface to which the ends of the elastic tubing anchor strap can attach, securely adhered to said i.v. tubing strap, and adhesively connected to said elastic tubing anchor strap; and
a soft surface, looped low pile material, square loop system, for providing a surface that will attach to the hook system allowing the elastic tubing anchor strap to secure the i.v. tubing, securely adhered to said elastic tubing anchor strap.

7. An i.v. tubing safety strap for securing i.v. tubing onto a patient without the use of adhesives; preventing pain and skin damage or exacerbation of pre-existing skin conditions, comprising:
an elastic/stretchable, rectangular elastic tubing anchor strap, for securing the i.v. tubing onto the strap providing stability and minimizing movement;
a circular, elastic/stretchable i.v. tubing strap, for providing a strap that is easy to apply onto a patient for the purpose of mounting i.v. tubing, threadably attached to said elastic tubing anchor strap;
a rough texture, rectangular, flexible hook system, for providing a surface to which the ends of the elastic tubing anchor strap can attach, securely adhered to said i.v. tubing strap, and adhesively connected to said elastic tubing anchor strap; and
a soft surface, looped low pile material, square loop system, for providing a surface that will attach to the hook system allowing the elastic tubing anchor strap to secure the i.v. tubing, securely adhered to said elastic tubing anchor strap.
